(19) United States
(12) Patent Application Publication
(10) Pub. No.: US 2025/0145581 A1
(43) Pub. Date: May 8, 2025

(54) PURIFICATION PROCESS FOR POLYMETHOXYLATED FLAVONOIDS AND COMPOSITIONS OBTAINED THEREFROM

(71) Applicant: Firmenich SA, Satigny (CH)

(72) Inventors: Wen-Juan XIANG, Shanghai (CN); Yi-Chun DING, Shanghai (CN)

(73) Assignee: Firmenich SA, Satigny (CH)

(21) Appl. No.: 18/833,258

(22) PCT Filed: Jan. 25, 2023
(86) PCT No.: PCT/EP2023/051798

§ 371 Date: Jul. 25, 2024

(30) Foreign Application Priority Data
Mar. 4, 2022 (WO) PCT/CN2022/079254
Mar. 31, 2022 (EP) 22165779.4

(51) Int. Cl. C07D 311/40; A23L 2/56; A23L 2/60; A23L 27/00; A23L 27/30

(52) U.S. Cl. CPC C07D 311/40; A23L 2/56; A23L 2/60; A23L 27/30; A23L 27/84

(57) ABSTRACT

The present disclosure relates generally to a process for obtaining a naturally derived composition rich in polymethoxylated flavonoids (PMFs). In some embodiments, the process results in a composition in which PMFs make up at least 90% by weight of the flavonoid content in the composition, based on the total weight of flavonoids in the composition. In certain aspects, the disclosure provides compositions resulting from the purification process and the use of such compositions to reduce bitterness, astringency, or sourness, or to enhance 0 sweetness or mouthfeel of an ingestible composition, such as a flavored food or beverage product. In some embodiments, the disclosure provides uses of the resulting composition to reduce the unpleasant taste of limonin, nomilin, or, one or more of which can be present at elevated levels in certain citrus products. naringin

### TECHNICAL FIELD

The present disclosure relates generally to a process for obtaining a naturally derived composition rich in polymethoxylated flavonoids (PMFs). In some embodiments, the process results in a composition in which PMFs make up at least 90% by weight of the flavonoid content in the composition, based on the total weight of flavonoids in the composition. In certain aspects, the disclosure provides compositions resulting from the purification process and the use of such compositions to reduce bitterness, astringency, or sourness, or to enhance sweetness or mouthfeel of an ingestible composition, such as a flavored food or beverage product. In some embodiments, the disclosure provides uses of the resulting composition to reduce the unpleasant taste of limonin, nomilin, or, one or more of which can be present at elevated levels in certain citrus products. naringin

### DESCRIPTION OF RELATED ART

The taste system provides sensory information about the chemical composition of the external world. Taste transduction is one of the more sophisticated forms of chemically triggered sensation in animals. Signaling of taste is found throughout the animal kingdom, from simple metazoans to the most complex of vertebrates. Mammals are believed to have five basic taste modalities: sweet, bitter, sour, salty, and umami.

Polymethoxylated flavonoids (PMFs) are a class of compounds that naturally occur in many citrus fruits. Such compounds have a wide array of applications. For example, PMFs may offer a number of benefits for health and wellness. These compounds can also be used to have certain beneficial effects on the taste of other products. For example, PCT Publication WO 2020/025639 describes, among other things, the use of certain PMFs for enhancing sweetness. As another example, PCT Publication WO 2020/074436 describes, among other things, the use of certain PMFs as mouthfeel enhancers and bitter maskers.

Extracting such compounds from citrus fruits in their substantially pure form is a cumbersome and costly process. Making such compounds via enzymatic conversion from other more readily available compounds in citrus fruits faces the same limitations. Therefore, the use of PMFs can be cost-prohibitive in certain applications. As an alternative, one may seek to extract a blend of flavonoids from the citrus fruit. But such methods, while providing improvements in terms of cost effectiveness, lead to compositions that contain unacceptable high concentrations of certain flavonoids that impart tastes that consumers generally perceive as unpleasant.

Therefore, there is a continuing need to develop cost-effective ways of obtaining PMFs without simultaneously obtaining other undesirable flavonoids.

### SUMMARY

The present disclosure relates to the discovery of a process for obtaining a blend of PMFs from a natural product with a concomitantly low concentration of other flavonoids that impart tastes that consumers generally perceive as unpleasant.

In a first aspect, the disclosure provides a process for concentrating polymethoxylated flavonoids, the process comprising: (a) providing an organic oil, which comprises polymethoxylated flavonoids and hydroxyl-substituted flavonoids, and wherein the concentration of polymethoxylated flavonoids in the organic oil is no more than 30% by weight, based on the total weight of the organic oil; (b) introducing an extraction medium to the organic oil to form a mixture having an aqueous phase and an organic phase, wherein the extraction medium comprises water and a C₁₋₆ alcohol; (c) separating at least a portion of the aqueous phase from the organic phase; and (d) generating two or more fractions of the separated aqueous phase using adsorption chromatography, wherein at least one of the two or more fractions comprises polymethoxylated flavonoids at a concentration of at least 85% by weight, on a dry weight basis.

In a second aspect, the disclosure provides a taste-modifying composition comprising one or more polymethoxylated flavonoids and one or more hydroxyl-substituted flavonoids. In some embodiments, the composition comprises polymethoxylated flavonoids or both polymethoxylated flavonoids and hydroxyl-substituted flavonoids at a concentration of at least 85% by weight, on a dry weight basis. In some embodiments, the weight ratio of polymethoxylated flavonoids to hydroxy-substituted flavonoids in the composition is at least 9:1. In certain related aspects, the taste-modifying composition is a product of the process of the first aspect, or any embodiments thereof.

In a third aspect, the disclosure provides uses of the taste-modifying composition of the second aspect, and aforementioned related aspects, to reduce a bitter taste of an ingestible composition. In certain related aspects, the disclosure provides methods of reducing a bitter taste of an ingestible composition, the method comprising introducing the taste-modifying composition of the second aspect, and aforementioned related aspects, to an ingestible composition. In some embodiments, the ingestible composition comprises one or more bitter compounds. In some embodiments, the one or more bitter compounds include limonoids, such as limonin, nomilin, nomilinic acid, or any combination thereof. In some embodiments, the one or more bitter compounds include naringin.

In a fourth aspect, the disclosure provides uses of the taste-modifying composition of the second aspect, and aforementioned related aspects, to reduce an astringent taste of an ingestible composition. In certain related aspects, the disclosure provides methods of reducing an astringent taste of an ingestible composition, the method comprising introducing the taste-modifying composition of the second aspect, and aforementioned related aspects, to an ingestible composition. In some embodiments, the ingestible composition comprises one or more astringent compounds.

In a fifth aspect, the disclosure provides uses of the taste-modifying composition of the second aspect, and aforementioned related aspects, to reduce a sour taste of an ingestible composition. In certain related aspects, the disclosure provides methods of reducing a sour taste of an ingestible composition, the method comprising introducing the taste-modifying composition of the second aspect, and aforementioned related aspects, to an ingestible composition. In some embodiments, the ingestible composition comprises one or more sour compounds, including, without limitation, certain organic acids, such as ascorbic acid, citric acid, lactic acid, malonic acid, malic acid, acetic acid, and the like.

In a sixth aspect, the disclosure provides uses of the taste-modifying composition of the second aspect, and aforementioned related aspects, to enhance a sweet taste of an ingestible composition. In certain related aspects, the disclosure provides methods of enhancing a sweet taste of an ingestible composition, the method comprising introducing the taste-modifying composition of the second aspect, and aforementioned related aspects, to an ingestible composition. In some embodiments, the ingestible composition comprises one or more sweeteners, including, without limitation, sucrose, fructose, glucose, galactose, lactose, allulose, erythritol, sucralose, saccharin, acesulfame potassium, aspartame, neotame, steviol glycosides (such as rebaudioside A, rebaudioside B, rebaudioside D, rebaudioside E, rebaudioside M, and the like), and megrosides (such as mogroside V, isomogroside V, siamenoside I, the α-1,6-isomer of siamenoside I, and the like).

In a seventh aspect, the disclosure provides ingestible compositions comprising the taste-modifying composition of the second aspect, and aforementioned related aspects. In some embodiments, the taste-modifying composition is present in the ingestible composition in a bitterness-reducing effective amount. In some embodiments thereof, the ingestible composition comprises one or more bitter compounds. In some embodiments thereof, the ingestible composition comprises one or more astringent compounds. In some embodiments thereof, the ingestible composition comprises one or more sour compounds. In some embodiments thereof, the ingestible composition comprises one or more sweeteners. In some embodiments thereof, the ingestible composition is a composition derived from citrus fruit. In some embodiments, the ingestible composition is a naturally occurring composition. In some other embodiments, the ingestible composition is a non-naturally occurring composition. In some embodiments, the bitter compounds are limonoids, such as limonin, nomilin, nomilinic acid, or any combination thereof. In some embodiments, the bitter compounds comprise naringin.

In an eighth aspect, the disclosure provides flavored products comprising an ingestible composition of the seventh aspect. In some embodiments, the flavored products are beverage products, such as soda, flavored water, tea, and the like. In some other embodiments, the flavored products are food products. In some embodiments, the flavored products are oral care products or chewing gum.

In a tenth aspect, the disclosure provides methods for reducing the bitterness of citrus compositions derived from citrus fruit having citrus greening disease, the method comprising: (a) deriving a citrus composition from citrus fruit, wherein at least a portion of the citrus fruit has citrus greening disease; and (b) introducing to the citrus composition a taste-modifying composition of the second aspect, or any aforementioned related aspects. In some embodiments, the citrus composition comprises an abnormally elevated concentration of one or more bitter compounds. In some embodiments, the bitter compounds are limonoids, such as limonin, nomilin, nomilinic acid, or any combination thereof. In some embodiments, the taste-modifying composition is derived from citrus waste.

Further aspects, and embodiments thereof, are set forth below in the Detailed Description, the Drawings, the Abstract, and the Claims.

### DETAILED DESCRIPTION

The following Detailed Description sets forth various aspects and embodiments provided herein. The description is to be read from the perspective of the person of ordinary skill in the relevant art. Therefore, information that is well known to such ordinarily skilled artisans is not necessarily included.

### Definitions

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

The term “bitter taste” refers to the activation of one or more T2R taste receptors by certain compounds in an ingestible composition. A “bitter receptor blocker” or “bitter blocker” refers to a compound that antagonizes the activation of one or more T2R receptors by a bitter compound. The reduction of bitter taste refers to the reduction (e.g., partial antagonism) of one or more T2R taste receptors that would otherwise be activated by one or more compounds in an ingestible composition.

The term “astringent taste” refers to a trigeminal effect that is perceived as dryness and as causing puckering. The reduction of astringent taste refers to the reduction of this trigeminal effect.

The term “sour taste” refers to the mildly unpleasant taste associated with organic acids, such as acetic acid (vinegar). The reduction of sour taste refers to the reduction of this unpleasant taste effect.

The term “sweet taste” refers to the activation of the T1R2/T1R3 taste receptors by certain compounds in an ingestible composition. A “sweet receptor enhancer” or “sweetness enhancer” refers to a compound that agonizes the activation of the T1R2/T1R3 taste receptor by a sweetener. The enhancement of sweet taste refers to the increase (e.g., agonism) of the T1R2/T1R3 taste receptor to a greater degree than it would otherwise be activated by one or more compounds in an ingestible composition.

The terms “polymethoxylated flavonoids” or “PMFs” or “PMF compounds” refer to flavonoid compounds having one or more methoxy substituents and no hydroxyl substituents. In some embodiments, the polymethoxylated flavonoids are polymethoxylated flavones, where the numberings follow the usual flavone numberings, as shown below:

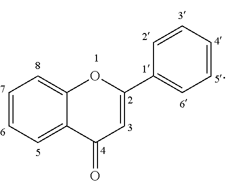 [SMILES: O=c1cc(-c2ccccc2)oc2ccccc12]

In some embodiments, the PMF compounds are one or more of the polymethoxylated flavone compounds set forth in Table 1.

TABLE 1
Examples of PMF Compounds

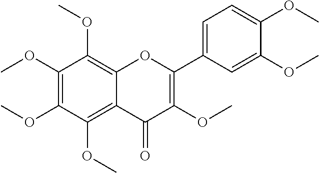

Heptamethoxyflavone (HMF)

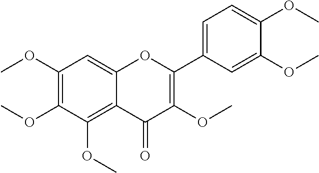

Hexamethoxyquercetogetin (HMQ)

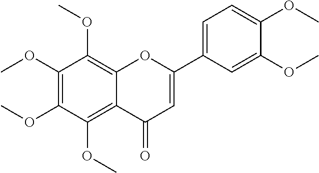

Nobiletin (NOB)

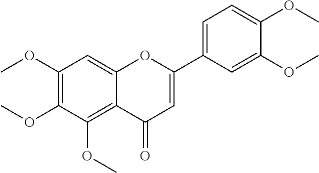

Sinensetin (SIN)

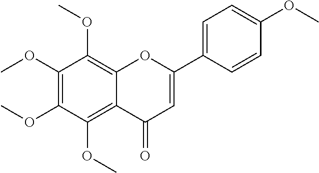

Tangeretin (TAN)

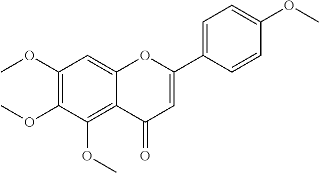

Tetramethoxyscutellarein (TMS)

The terms “hydroxylated flavonoids,” “hydroxy-substituted flavonoids,” “hydroxyl-substituted flavonoids,” or “HFs” or “HF compounds” refer to flavonoid compounds having at least one hydroxyl substituent. In some embodiments, the hydroxylated flavonoids are hydroxylated flavones. Non-limiting examples include 5OHSIN (CAS No. 21763-80-4), 5OHHMQ (CAS No. 479-90-3), 5OHNOB (CAS No. 2174-59-6), 5OHTMS (CAS No. 19103-54-9), 5OHHMF (CAS No. 1176-88-1), and 5OHTAN (CAS No. 2798-20-1).

As used herein, the singular forms “a,” “an,” and “the” include plural referents unless the context clearly dictates otherwise. For example, reference to “a substituent” encompasses a single substituent as well as two or more substituents, and the like.

As used herein, “hydroxy” refers to an —OH substituent, and “methoxy” refers to an —OCH₃ substituent.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

Chemical structures are often shown using the “skeletal” format, such that carbon atoms are not explicitly shown, and hydrogen atoms attached to carbon atoms are omitted entirely. For example, the structure

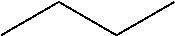

represents butane (i.e., n-butane). Furthermore, aromatic groups, such as benzene, are represented by showing one of the contributing resonance structures. For example, the structure

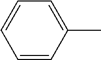

represents toluene.

Other terms are defined in other portions of this description, even though not included in this subsection.

### PMF Concentration Process

In certain aspects, the disclosure provides a process for concentrating polymethoxylated flavonoids, the process comprising: (a) providing an organic oil, which comprises polymethoxylated flavonoids and hydroxyl-substituted flavonoids; (b) introducing an extraction medium to the organic oil to form a mixture having an aqueous phase and an organic phase, wherein the extraction medium comprises water and a C₁₋₆ alcohol; (c) separating at least a portion of the aqueous phase from the organic phase; and (d) generating two or more fractions of the separated aqueous phase using adsorption chromatography, wherein at least one of the two or more fractions comprises polymethoxylated flavonoids or both polymethoxylated flavonoids and hydroxyl-substituted flavonoids at a concentration of at least 85% by weight, on a dry weight basis.

As noted above, the process involves extracting and concentrating the PMF compounds from an organic oil. The organic oil can be obtained from any suitable source. For example, in some embodiments, the organic oil is a citrus oil. Such citrus oils can be obtained in any suitable way. For example, in some embodiments, the citrus oil is derived from extraction and/or distillation of oils from citrus fruit, including citrus peel. Any suitable citrus fruit can be used, including, without limitation, orange, lemon, grapefruit, lime, kumquat, pomelo, tangelo, ugli, tangerines, yuzu, or any combination thereof. In some embodiments, the citrus oil is orange peel oil. The organic oil generally contains a variety of other compounds besides PMFs, including HFs and a variety of other organic compounds. In some embodiments, the concentration of PMFs in the organic oil is no more than 15% by weight, or no more than 20% by weight, or no more than 25% by weight, or no more than 30% by weight, or no more than 35% by weight, based on the total weight of the organic oil. The organic oil also contains HFs. In some embodiments, the weight ratio of PMFs to HFs in the organic oil is no more than 12:1, or no more than 14:1, or no more than 16:1, or no more than 18:1. In some embodiments, the concentration of HFs in the organic oil is at least 0.5% by weight, based on the total weight of the organic oil.

The concentration process disclosed herein comprises introducing an extraction medium to the organic oil to form a mixture having an aqueous phase and an organic phase. In general, the extraction medium comprises one or more solvents that will form a separate phase from the organic oil. Such solvents include water and other water-miscible solvents, such as acetone, acetonitrile, tetrahydrofuran, acetaldehyde, acetone, diethanolamine, dimethylformamide, 1.4-dioxane, ethylamine, furfuryl alcohol, diethylene glycol, triethylene glycol, and other C₁₋₅ alcohols. Examples of such C₁₋₅ alcohols include, without limitation, methanol, ethanol, 1-propanol, 2-propanol, ethylene glycol, 1,2-propanediol, 1,3-propanediol, 1,2-butanediol, 1,3-butanediol, 1,4-butanediol, 1,5-pentanediol, and glycerol. In some embodiments, the extraction medium comprises water and one or more C₁₋₅ alcohols. In some embodiments, the extraction medium comprises water and ethanol. In some such embodiments, the concentration of ethanol in the extraction medium ranges from 5% by weight to 65% by weight, or from 10% by weight to 60% by weight, or from 20% by weight to 60% by weight, or from 30% by weight to 60% by weight. In some such embodiments, the concentration of water in the extraction medium ranges from 35% by weight to 95% by weight, or from 40% by weight to 90% by weight, or from 40% by weight to 80% by weight, or from 40% by weight to 70% by weight.

By contacting the organic oil with the extraction medium, certain compounds in the organic oil are extracted into the aqueous phase. For example, in some embodiments, the PMFs make up at least 50% by weight, or at least 55% by weight, or at least 60% by weight of the flavonoids present in the aqueous phase, based on total flavonoid content of the aqueous phase. In some embodiments, the weight ratio of PMFs to HFs in the aqueous phase is no more than 12:1, or no more than 14:1, or no more than 16:1, or no more than 18:1. In some embodiments, the concentration of HFs in the aqueous phase is at least 5% by weight, based on total flavonoid content of the aqueous phase.

The concentration process disclosed herein comprises separating at least a portion of the aqueous phase from the organic phase of the mixture formed after the extraction medium is introduced to the organic oil. The separating can be carried out by any suitable means. In general, the two phases are permitted to separate, so that a higher proportion of the aqueous phase can be separated. In some embodiments, the separating occurs in a single removal step. In other embodiments, the separating occurs in two or more removal steps.

The concentration process disclosed herein comprises generating two or more fractions of the separated aqueous phase using adsorption chromatography. In some embodiments, at least three fractions, or at least four fractions, of at least five fractions, or at least six fractions, or at least seven fractions, or at least eight fractions, or at least nine fractions, or at least ten fractions.

The fractions can be generated by any suitable means. In some embodiments, the fractions are generated by chromatography, where the separated aqueous phase is eluted in a mobile phase in the presence of a stationary phase. In general, different components of the eluted composition (for example, the separated aqueous phase) will have different relative affinities for the aqueous phase and the mobile phase, such that, as fractions are collected throughout the course of the elution, the fractions tend to contain concentrated amounts of compounds that have similar relative affinities. Any suitable chromatographic technique can be used. In some embodiments, the fractions are generated by adsorption chromatography (or liquid-solid chromatography), where the mobile phase is a liquid and the stationary phase is the surface of a solid, such as a solid support.

In embodiments where adsorption chromatography is used, any suitable mobile phase and solid support can be used, according to the knowledge of skilled artisans. In some embodiments, the stationary phase is a macroporous resin that is non-polar or only moderately polar. Such a stationary phase will tend to delay the progress of more nonpolar (or hydrophobic) compounds, such that these compounds are concentrated in later-collected fractions.

In some embodiments, the mobile phase is a polar medium, such as an aqueous medium. In some embodiments, the mobile phase comprises water and one or more water-miscible solvents. Such water-miscible solvents include, but are not limited to, acetone, acetonitrile, tetrahydrofuran, acetaldehyde, acetone, diethanolamine, dimethylformamide, 1.4-dioxane, ethylamine, furfuryl alcohol, diethylene glycol, triethylene glycol, and other C₁₋₅ alcohols. Examples of such C₁₋₅ alcohols include, without limitation, methanol, ethanol, 1-propanol, 2-propanol, ethylene glycol, 1,2-propanediol, 1,3-propanediol, 1,2-butanediol, 1,3-butanediol, 1,4-butanediol, 1,5-pentanediol, and glycerol. In some embodiments, the mobile phase comprises water and one or more C₁₋₅ alcohols. In some embodiments, the mobile phase comprises water and ethanol. In some such embodiments, the concentration of ethanol in the mobile phase ranges from 5% by weight to 65% by weight, or from 10% by weight to 60% by weight, or from 20% by weight to 60% by weight, or from 30% by weight to 60% by weight. In some such embodiments, the concentration of water in the mobile phase ranges from 35% by weight to 95% by weight, or from 40% by weight to 90% by weight, or from 40% by weight to 80% by weight, or from 40% by weight to 70% by weight.

In some embodiments, at least one of the two or more fractions comprises PMFs and HFs at a concentration of at least 80% by weight, or at least 85% by weight, or at least 87% by weight, on a dry weight basis. In some embodiments, the weight ratio of PMFs to HFs in the at least one of the two or more fractions is at least 9:1, at least 10:1, at least 11:1, at least 12:1, or at least 15:1, or at least 17:1, or at least 19:1. In some embodiments, the concentration of HFs in the at least one of the two or more fractions is no more than 10% by weight, no more than 9% by weight no more than 8% by weight no more than 7% by weight, or no more than 6% by weight, or no more than 5% by weight, on a dry weight basis.

In some embodiments, the solvent is partially or fully removed from the at least one of the two or more fractions. Solvent removal can be accomplished by any suitable means, include evaporation, drying (through moderate heating), vacuum-assisted methods, and the like. Such removal yields either a highly concentrated PMF composition or a dry solid containing mostly PMFs.

In some embodiments, the at least one of the two or more fractions, which can be optionally dried or concentrated through solvent removal, is washed with an aqueous alkaline solution. Any suitable water-soluble base can be used to make the alkaline wash solution. Suitable bases include, without limitation, sodium hydroxide, potassium hydroxide, sodium carbonate, sodium bicarbonate, calcium oxide, calcium carbonate, potassium carbonate, or any combinations thereof. In some embodiments, the alkaline wash solution can also contain certain metal compounds, such as ferric chloride.

The base can be any suitable concentration, for example, around 5% by weight of the wash solution, based on the total weight of the wash solution.

In some embodiments, washing the at least one of the two or more fractions, which can be optionally dried or concentrated through solvent removal, forms an insoluble solid in the aqueous medium, which can be collected and dried. This insoluble solid is typically has a high concentration of PMFs. For example, in some embodiments, the insoluble solid comprises PMFs and HFs at a concentration of at least 85% by weight, or at least 87% by weight, or at least 90% by weight, on a dry weight basis. In some embodiments, the weight ratio of PMFs to HFs in the insoluble solid is at least 9:1, at least 10:1, at least 11:1 at least 12:1, or at least 15:1, or at least 17:1, or at least 19:1. In some embodiments, the concentration of HFs in the insoluble solid is no more than 10% by weight, no more than 9% by weight, no more than 8% by weight, or no more than 7% by weight, or no more than 6% by weight, or no more than 5% by weight, on a dry weight basis.

Additional steps can be applied to the recovered solid to prepare it for storage and to prevent oxidation, thermal breakdown, or breakdown by light.

### Taste-Modifying Compositions

The process set forth above can lead to the formation of highly concentrated compositions of PMFs, as described in certain embodiments above. In some embodiments, the process set forth above leads to PMF-containing compositions having reduced off note intensity.

This, in a certain aspects, the disclosure provides a taste-modifying composition comprising one or more PMFs and one or more HFs. In some embodiments, the PMFs are flavone compounds. In some further such embodiments, the PMFs are one or more compounds selected from the group consisting of the six compounds in Table 1, or any combinations thereof.

In some embodiments, the taste-modifying composition comprises PMFs or both PMFs and HFs at a concentration of at least 85% by weight, or at least 87% by weight, or at least 90% by weight, on a dry weight basis. In some embodiments, the weight ratio of PMFs to HFs in the taste-modifying composition is at least 9:1, at least 10:1, at least 11:1, at least 12:1, or at least 15:1, or at least 17:1, or at least 19:1. In some embodiments, the concentration of HFs in the taste-modifying composition is no more than 10% by weight, no more than 9% by weight, no more than 8% by weight, no more than 7% by weight, or no more than 6% by weight, or no more than 5% by weight, on a dry weight basis (excluding the weight of a bulking agent, if present, in a solid composition).

The taste-modifying composition can exist in any suitable form. For example, in some embodiments, the taste-modifying composition is a solid, such as a solid in the form of a powder, granules, and the like. In some such embodiments, other ingredients can be added, such as bulking agents, desiccants, and the like. In some embodiments, the taste-modifying composition is a liquid, where a liquid carrier, such an aqueous carrier, is introduced to dissolve or suspend the PMF compounds.

In some embodiments, the taste-modifying composition is the product of the processes provided above, or any embodiments thereof.

### Uses and Methods

In certain aspects, the disclosure provides uses of a taste-modifying composition of any of the foregoing aspects and embodiments to reduce a bitter taste of an ingestible composition. In some embodiments thereof, the ingestible composition comprises one or more bitter compounds. In some embodiments thereof, the ingestible composition is a composition derived from citrus fruit. In some embodiments, the one or more bitter compounds include limonoids, such as limonin, nomilin, nomilinic acid, or any combination thereof. In some embodiments, the one or more bitter compounds include naringin. In certain related aspects, the disclosure provides methods of reducing a bitter taste of an ingestible composition, the method comprising introducing a taste-modifying composition of any of the foregoing aspects and embodiments to the ingestible composition.

In certain aspects, the disclosure provides uses of a taste-modifying composition of any of the foregoing aspects and embodiments to reduce an astringent taste of an ingestible composition. In some embodiments thereof, the ingestible composition comprises one or more astringent compounds. In some embodiments thereof, the ingestible composition is a composition derived from citrus fruit. In certain related aspects, the disclosure provides methods of reducing an astringent taste of an ingestible composition, the method comprising introducing a taste-modifying composition of any of the foregoing aspects and embodiments to the ingestible composition.

In certain aspects, the disclosure provides uses of a taste-modifying composition of any of the foregoing aspects and embodiments to reduce a sour taste of an ingestible composition. In some embodiments thereof, the ingestible composition comprises one or more sour compounds, such as an organic acid. Examples of such organic acids include ascorbic acid, acetic acid, citric acid, lactic acid, malic acid, malonic acid, and the like. In some embodiments thereof, the ingestible composition is a composition derived from citrus fruit. In some embodiments, the ingestible composition is a fermented beverage, such as kombucha. In certain related aspects, the disclosure provides methods of reducing a sour taste of an ingestible composition, the method comprising introducing a taste-modifying composition of any of the foregoing aspects and embodiments to the ingestible composition.

In certain aspects, the disclosure provides uses of a taste-modifying composition of any of the foregoing aspects and embodiments to enhance a sweet taste of an ingestible composition. In some embodiments thereof, the ingestible composition comprises one or more sweeteners. Suitable sweeteners include natural sweeteners, such as glucose, sucrose, fructose, and the like, as well as high-potency sweeteners such as aspartame, neotame, sucralose, saccharin, acesulfame potassium, rebaudiosides, mogrosides, and the like. In some embodiments, the ingestible composition is a composition derived from citrus fruit. In certain related aspects, the disclosure provides methods of enhancing a sweet taste of an ingestible composition, the method comprising introducing a taste-modifying composition of any of the foregoing aspects and embodiments to the ingestible composition.

In certain aspects, the disclosure provides uses of a taste-modifying composition of any of the foregoing aspects and embodiments to enhance a mouthfeel of an ingestible composition. In some embodiments thereof, the ingestible composition comprises one or more sweeteners. Suitable sweeteners include natural sweeteners, such as glucose, sucrose, fructose, and the like, as well as high-potency sweeteners such as aspartame, neotame, sucralose, saccharin, acesulfame potassium, rebaudiosides, mogrosides, and the like. In some embodiments, the ingestible composition is a composition derived from citrus fruit. In certain related aspects, the disclosure provides methods of enhancing a mouthfeel of an ingestible composition, the method comprising introducing a taste-modifying composition of any of the foregoing aspects and embodiments to the ingestible composition.

The foregoing uses and methods generally involve the use of the PMF-containing taste-modifying composition in an ingestible composition containing one or more additional ingredients. In certain embodiments of any aspects and embodiments set forth herein that refer to an ingestible composition, the ingestible composition is a non-naturally-occurring product, such as a composition specifically manufactured for the production of a flavored product, such as food or beverage product.

In certain embodiments, the disclosure provides ingestible compositions comprising a taste-modifying composition of any of the foregoing aspects and embodiments. In some embodiments, the taste-modifying composition is present in the ingestible composition in a concentration sufficient to reduce bitterness, reduce astringency, reduce sourness, enhance sweetness, or enhance mouthfeel. In some embodiments thereof, the ingestible composition comprises one or more bitter compounds. In some embodiments thereof, the ingestible composition comprises one or more astringent compounds. In some embodiments thereof, the ingestible composition comprises one or more sour compounds. In some embodiments thereof, the ingestible composition comprises one or more sweeteners. In some embodiments thereof, the ingestible composition is a composition derived from citrus fruit. In some embodiments, the ingestible composition is a naturally occurring composition. In some other embodiments, the ingestible composition is a non-naturally occurring composition. In some embodiments, the bitter compounds are limonoids, such as limonin, nomilin, nomilinic acid, or any combination thereof. In some embodiments, the bitter compounds comprise naringin.

The taste-modifying composition can be present in the ingestible composition in any suitable concentration. For example, in some embodiments, the taste-modifying composition is present in the ingestible composition at a concentration sufficient for the concentration of PMF compounds in the ingestible composition to range from 0.01 ppm to 1000 ppm, or from 0.01 ppm to 900 ppm, or from 0.01 ppm to 800 ppm, or from 0.01 ppm to 700 ppm, or from 0.01 ppm to 600 ppm, or from 0.1 ppm to 500 ppm, or from 0.1 ppm to 400 ppm, or from 0.1 ppm to 300 ppm, or from 0.1 ppm to 200 ppm, or from 1 ppm to 100 ppm, or from 1 ppm to 80 ppm, or from 1 ppm to 60 ppm, or from 1 ppm to 50 ppm, or from 1 ppm to 40 ppm.

The ingestible compositions set forth herein, whether as a composition or as part of a use or method, in some embodiments, comprise one or more bitter compounds. The bitter compounds mentioned above can be any suitable bitter compounds commonly found in natural food products. In some embodiments, the bitter compound are compounds extracted of a plant from the Meliaceae family or a plant from the Rutacaea family. In some further embodiments, the bitter compound is an extract of a plant from the Rutacaea family and the Citrus genus, such as an extract from the tree or fruit of orange, lemon, grapefruit, lime, kumquat, pomelo, tangelo, ugli, tangerine, or yuzu. In some embodiments, the bitter compound is a triterpene. In some further embodiments, the bitter compound is a tetranortriterpenoid. In some further embodiments, the bitter compound is a limonoid. In some such embodiments, the bitter compound is limonin, nomilin, nomilic acid, azadirachtin, or any combination thereof. In some embodiments, the bitter compound is limonin. In some embodiments, the bitter compound is nomilin. In some embodiments, the bitter compound is naringin.

In certain other embodiments, the ingestible composition comprises bitter compounds and the taste-modifying composition. In some embodiments, the concentration of the PMF compounds in the ingestible composition is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The bitter compounds include, but are not limited to, active pharmaceutical ingredients (APIs), tannins (such as those in coffee, tea, wine), caffeine, quinine, catechins, polyphenols, potassium chloride, menthol, or plant starches or proteins, algal proteins or starches, fungal proteins or starches, alcohol, and any combination thereof.

In certain other embodiments, the ingestible composition comprises sour compounds and the taste-modifying composition. In some embodiments, the concentration of the PMF compounds is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The sour compounds include, but are not limited to, organic acids, such as acetic acid, malonic acid, citric acid, and the like.

In some instances, one may be able to reduce the amount of sweetener in a product by reducing bitter taste or enhancing sweetness.

In certain other embodiments, the ingestible composition comprises sucrose and the taste-modifying composition. In some such embodiments, the introduction of the PMF compounds permits one to use less sucrose (such as more than 10% less, more than 20% less, more than 30% less, more than 40% less, more than 50% less, more than 60% less, or more than 70% less) and still achieve a level of sweetness characteristic of a comparable product that employs more sucrose. In some embodiments, the concentration of the PMF compounds is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The sucrose can be introduced in any suitable form, such as natural syrups (cane syrup) and the like.

In certain other embodiments, the ingestible composition comprises fructose (for example, in the form of high-fructose corn syrup) and the taste-modifying composition. In some such embodiments, the introduction of the PMF compounds permits one to use less fructose (such as more than 10% less, more than 20% less, more than 30% less, more than 40% less, more than 50% less, more than 60% less, or more than 70% less) and still achieve a level of sweetness characteristic of a comparable product that employs more fructose. In some embodiments, the concentration of the PMF compounds is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. In some embodiments, the ingestible composition is a food product, such as any of those specifically listed below. In other embodiments, the ingestible composition is a beverage product, such as a soda, and the like. The fructose can be supplied in any suitable form, such as natural syrups, high-fructose corn syrup, and the like.

In certain other embodiments, the ingestible composition comprises D-glucose and the taste-modifying composition. In some such embodiments, the introduction of the PMF compounds permits one to use less D-glucose (such as more than 10% less, more than 20% less, more than 30% less, more than 40% less, more than 50% less, more than 60% less, or more than 70% less) and still achieve a level of sweetness characteristic of a comparable product that employs more glucose. In some embodiments, the concentration of PMF compounds is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. The glucose can be introduced in any suitable form, such as natural syrups and the like.

In certain other embodiments, the ingestible composition comprises sucralose and the taste-modifying composition. In some such embodiments, the introduction of the PMF compounds permits one to use less sucralose (such as more than 10% less, more than 20% less, more than 30% less, more than 40% less, more than 50% less, more than 60% less, or more than 70% less) and still achieve a level of sweetness characteristic of a comparable product that employs more sucralose. In some embodiments, the concentration of the PMF compounds is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain other embodiments, the ingestible composition comprises rebaudiosides (such as rebaudioside A, rebaudioside B, rebaudioside D, rebaudioside E, rebaudioside M, or any combination thereof) and the taste-modifying composition. In some such embodiments, the introduction of the PMF compounds permits one to use less rebaudioside (such as more than 10% less, more than 20% less, more than 30% less, more than 40% less, more than 50% less, more than 60% less, or more than 70% less) and still achieve a level of sweetness characteristic of a comparable product that employs more rebaudioside. In some embodiments, the concentration of the PMF compounds is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain other embodiments, the ingestible composition comprises acesulfame potassium and the taste-modifying composition. In some such embodiments, the introduction of the PMF compounds permits one to use less acesulfame K (such as more than 10% less, more than 20% less, more than 30% less, more than 40% less, more than 50% less, more than 60% less, or more than 70% less) and still achieve a level of sweetness characteristic of a comparable product that employs more acesulfame K. In some embodiments, the concentration of the PMF compounds is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain other embodiments, the ingestible composition comprises allulose and the taste-modifying composition. In some such embodiments, the introduction of the PMF compounds permits one to use less allulose (such as more than 10% less, more than 20% less, more than 30% less, more than 40% less, more than 50% less, more than 60% less, or more than 70% less) and still achieve a level of sweetness characteristic of a comparable product that employs more allulose. In some embodiments, the concentration of the PMF compounds is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain other embodiments, the ingestible composition comprises erythritol and the taste-modifying composition. In some such embodiments, the introduction of the PMF compounds permits one to use less erythritol (such as more than 10% less, more than 20% less, more than 30% less, more than 40% less, more than 50% less, more than 60% less, or more than 70% less) and still achieve a level of sweetness characteristic of a comparable product that employs more erythritol. In some embodiments, the concentration of the PMF compounds is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain other embodiments, the ingestible composition comprises aspartame or neotame and the taste-modifying composition. In some such embodiments, the introduction of the PMF compounds permits one to use less aspartame or neotame (such as more than 10% less, more than 20% less, more than 30% less, more than 40% less, more than 50% less, more than 60% less, or more than 70% less) and still achieve a level of sweetness characteristic of a comparable product that employs more aspartame or neotame. In some embodiments, the concentration of the PMF compounds is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like.

In certain other embodiments, the ingestible composition comprises a mogroside (such as mogroside III, mogroside IV, mogroside V, siamenoside, isomogroside V, mogroside IVE, isomogroside V, mogroside IIIE, 11-oxomogroside V, the alpha isomer of siamenoside I, and any combinations thereof) and the taste-modifying composition. In some such embodiments, the introduction of the PMF compounds permits one to use less a mogroside (such as more than 10% less, more than 20% less, more than 30% less, more than 40% less, more than 50% less, more than 60% less, or more than 70% less) and still achieve a level of sweetness characteristic of a comparable product that employs more mogroside. In some embodiments, the concentration of the PMF compounds is no more than 1000 ppm, or no more than 900 ppm, or no more than 800 ppm, or no more than 700 ppm, or no more than 600 ppm, or no more than 500 ppm, or no more than 400 ppm, or no more than 300 ppm, or no more than 200 ppm, or no more than 100 ppm, or no more than 50 ppm, or no more than 25 ppm, or no more than 10 ppm. Such ingestible compositions can be in any suitable form. In some embodiments, the ingestible composition is a food product, such as any of those specifically listed below. In other embodiments, the ingestible composition is a beverage product, such as a soda, and the like. Additional mogroside compounds that may be suitably used are described in U.S. Patent Application Publication No. 2017/0119032.

Thus, in some embodiments, the compositions set forth in any of the foregoing aspects (including in any uses or methods), comprise the taste-modifying composition and one or more sweeteners. In some embodiments, the composition further comprises a vehicle. In some embodiments, the vehicle is water. In some embodiments, the PMF compounds are present at a concentration at or below its sweetness recognition threshold.

For example, in some embodiments, the sweetener is present in an amount from about 0.1% to about 12% by weight. In some embodiments, the sweetener is present in an amount from about 0.2% to about 10% by weight. In some embodiments, the sweetener is present in an amount from about 0.3% to about 8% by weight. In some embodiments, the sweetener is present in an amount from about 0.4% to about 6% by weight. In some embodiments, the sweetener is present in an amount from about 0.5% to about 5% by weight. In some embodiments, the sweetener is present in an amount from about 1% to about 2% by weight. In some embodiments, the sweetener is present in an amount from about 0.1% to about 5% by weight. In some embodiments, the sweetener is present in an amount from about 0.1% to about 4% by weight. In some embodiments, the sweetener is present in an amount from about 0.1% to about 3% by weight. In some embodiments, the sweetener is present in an amount from about 0.1% to about 2% by weight. In some embodiments, the sweetener is present in an amount from about 0.1% to about 1% by weight. In some embodiments, the sweetener is present in an amount from about 0.1% to about 0.5% by weight. In some embodiments, the sweetener is present in an amount from about 0.5% to about 10% by weight. In some embodiments, the sweetener is present in an amount from about 2% to about 8% by weight. In some further embodiments of the embodiments set forth in this paragraph, the sweetener is sucrose, fructose, glucose, xylitol, erythritol, or combinations thereof.

In some other embodiments, the sweetener is present in an amount from 10 ppm to 1000 ppm. In some embodiments, the sweetener is present in an amount from 20 ppm to 800 ppm. In some embodiments, the sweetener is present in an amount from 30 ppm to 600 ppm. In some embodiments, the sweetener is present in an amount from 40 ppm to 500 ppm. In some embodiments, the sweetener is present in an amount from 50 ppm to 400 ppm. In some embodiments, the sweetener is present in an amount from 50 ppm to 300 ppm. In some embodiments, the sweetener is present in an amount from 50 ppm to 200 ppm. In some embodiments, the sweetener is present in an amount from 50 ppm to 150 ppm. In some further embodiments of the embodiments set forth in this paragraph, the sweetener is a steviol glycoside, a mogroside, a derivative of either of the foregoing, such as glycoside derivatives (e.g., glucosylates), or any combination thereof.

The compositions can include any suitable sweeteners or combination of sweeteners. In some embodiments, the sweetener is a common saccharide sweeteners, such as sucrose, fructose, glucose, and sweetener compositions comprising natural sugars, such as corn syrup (including high fructose corn syrup) or other syrups or sweetener concentrates derived from natural fruit and vegetable sources. In some embodiments, the sweetener is sucrose, fructose, or a combination thereof. In some embodiments, the sweetener is sucrose. In some other embodiments, the sweetener is selected from rare natural sugars including D-allose, D-psicose, L-ribose, D-tagatose, L-glucose, L-fucose, L-arbinose, D-turanose, and D-leucrose. In some embodiments, the sweetener is selected from semi-synthetic “sugar alcohol” sweeteners such as erythritol, isomalt, lactitol, mannitol, sorbitol, xylitol, maltodextrin, and the like. In some embodiments, the sweetener is selected from artificial sweeteners such as aspartame, saccharin, acesulfame-K, cyclamate, sucralose, and alitame. In some embodiments, the sweetener is selected from the group consisting of cyclamic acid, mogroside, tagatose, maltose, galactose, mannose, sucrose, fructose, lactose, neotame and other aspartame derivatives, glucose, D-tryptophan, glycine, maltitol, lactitol, isomalt, hydrogenated glucose syrup (HGS), hydrogenated starch hydrolyzate (HSH), stevioside, rebaudioside A, other sweet Stevia-based glycosides, chemically modified steviol glycosides (such as glucosylated steviol glycosides), mogrosides, chemically modified mogrosides (such as glucosylated mogrosides), carrelame and other guanidine-based sweeteners. In some embodiments, the sweetener is a combination of two or more of the sweeteners set forth in this paragraph. In some embodiments, the sweetener may combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a sugar. In some embodiments, the sugar is cane sugar. In some embodiments, the sugar is beet sugar. In some embodiments, the sugar may be sucrose, fructose, glucose or combinations thereof. In some embodiments, the sugar may be sucrose. In some embodiments, the sugar may be a combination of fructose and glucose.

The sweetener can also include, for example, sweetener compositions comprising one or more natural or synthetic carbohydrate, such as corn syrup, high fructose corn syrup, high maltose corn syrup, glucose syrup, sucralose syrup, hydrogenated glucose syrup (HGS), hydrogenated starch hydrolyzate (HSH), or other syrups or sweetener concentrates derived from natural fruit and vegetable sources, or semi-synthetic “sugar alcohol” sweeteners such as polyols. Non-limiting examples of polyols in some embodiments include erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, isomaltulose, maltodextrin, and the like, and sugar alcohols or any other carbohydrates or combinations thereof capable of being reduced which do not adversely affect taste.

The sweetener may be a natural or synthetic sweetener that includes, but is not limited to, agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green stevia powder, stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, glycyrrhetic acid monoglucuronide, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside IV, mogroside V, and neomogroside), mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

In still other embodiments, the sweetener can be a chemically or enzymatically modified natural high potency sweetener. Modified natural high potency sweeteners include glycosylated natural high potency sweetener such as glucosyl-, galactosyl-, or fructosyl-derivatives containing 1-50 glycosidic residues. Glycosylated natural high potency sweeteners may be prepared by enzymatic transglycosylation reaction catalyzed by various enzymes possessing transglycosylating activity. In some embodiments, the modified sweetener can be substituted or unsubstituted.

Additional sweeteners also include combinations of any two or more of any of the aforementioned sweeteners. In some embodiments, the sweetener may comprise combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a caloric sweetener, such as sucrose, fructose, xylitol, erythritol, or combinations thereof. In some embodiments, the ingestible compositions are free (or, in some embodiments) substantially free of stevia-derived sweeteners, such as steviol glycosides, glucosylated steviol glycosides, or rebaudiosides. For example, in some embodiments, the ingestible compositions are either free of stevia-derived sweeteners or comprise stevia-derived sweeteners in a concentration of no more than 1000 ppm, or no more than 500 ppm, or no more than 200 ppm, or no more than 100 ppm, or no more than 50 ppm, or no more than 20 ppm, or no more than 10 ppm, or no more than 5 ppm, or no more than 3 ppm, or no more than 1 ppm.

The ingestible compositions or sweetener concentrates can, in certain embodiments, comprise any additional ingredients or combination of ingredients as are commonly used in food and beverage products, including, but not limited to:
acids, including, for example citric acid, phosphoric acid, ascorbic acid, sodium acid sulfate, lactic acid, or tartaric acid;
bitter ingredients, including, for example caffeine, quinine, green tea, catechins, polyphenols, green robusta coffee extract, green coffee extract, potassium chloride, menthol, or proteins (such as proteins and protein isolates derived from plants, algae, or fungi);
coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide;
preservatives, including, for example sodium benzoate, potassium benzoate, potassium sorbate, sodium metabisulfate, sorbic acid, or benzoic acid;
antioxidants including, for example ascorbic acid, calcium disodium EDTA, alpha tocopherols, mixed tocopherols, rosemary extract, grape seed extract, resveratrol, or sodium hexametaphosphate;
vitamins or functional ingredients including, for example resveratrol, Co-Q10, omega 3 fatty acids, theanine, choline chloride (citocoline), fibersol, inulin (chicory root), taurine, panax ginseng extract, guanana extract, ginger extract, L-phenylalanine, L-carnitine, L-tartrate, D-glucoronolactone, inositol, bioflavonoids, Echinacea, ginko biloba, yerba mate, flax seed oil, garcinia cambogia rind extract, white tea extract, ribose, milk thistle extract, grape seed extract, pyrodixine HCl (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulphate;
clouding agents, including, for example ester gun, brominated vegetable oil (BVO), or sucrose acetate isobutyrate (SAIB);
buffers, including, for example sodium citrate, potassium citrate, or salt;
flavors, including, for example propylene glycol, ethyl alcohol, glycerine, gum Arabic (gum acacia), maltodextrin, modified corn starch, dextrose, natural flavor, natural flavor with other natural flavors (natural flavor WONF), natural and artificial flavors, artificial flavor, silicon dioxide, magnesium carbonate, or tricalcium phosphate; or
starches and stabilizers, including, for example pectin, xanthan gum, carboxylmethylcellulose (CMC), polysorbate 60, polysorbate 80, medium chain triglycerides, cellulose gel, cellulose gum, sodium caseinate, modified food starch, gum Arabic (gum acacia), inulin, or carrageenan.

The ingestible compositions can have any suitable pH. In some embodiments, the taste-modifying compositions disclosed herein enhance the sweetness of a sweetener under a broad range of pH, e.g., from lower pH to neutral pH. The lower and neutral pH includes, but is not limited to, a pH from about 2.5 to about 8.5; from about 3.0 to about 8.0; from about 3.5 to about 7.5; and from about 4.0 to about 7. In certain embodiments, compounds as disclosed and described herein, individually or in combination, can enhance the perceived sweetness of a fixed concentration of a sweetener in taste tests at a compound concentration of about 50 μM, 40 μM, 30 μM, 20 μM, or 10 μM at both low to neutral pH value. In certain embodiments, the enhancement factor of the compounds as disclosed and described herein, individually or in combination, at the lower pH is substantially similar to the enhancement factor of the compounds at neutral pH. Such consistent sweet enhancing property under a broad range of pH allow a broad use in a wide variety of foods and beverages of the compounds as disclosed and described herein, individually or in combination.

The ingestible compositions set forth according to any of the foregoing embodiments, also include, in certain embodiments, one or more additional compounds, such as compounds that enhance sweetness (e.g., hesperetin, hesperetin dihydrochalcone, neohesperidin dihydrochalcone, neohesperitin, phloretin, naringenin, eriodictyol, homoeriodictyol, trilobatin, hesperitin dihydrochalcone glucoside, 3-hydroxybenzoic acid, glucosylated steviol glycosides, (2R,3R)-3-acetoxy-5,7,4′-trihydroxyflavanone, (2R,3R)-3-acetoxy-5,7,3′-trihydroxy-4′-methoxyflavanone, etc.), compounds that block bitterness, compounds that enhance umami, compounds that reduce sourness or licorice taste, compounds that enhance saltiness, compounds that enhance a cooling effect, or any combinations of the foregoing.

Thus, in some embodiments, ingestible compositions disclosed herein comprise a taste-modifying composition according to any of the embodiments or combination of embodiments set forth above, are combined with one or more sweetness enhancing compounds. Such sweetness enhancing compounds include, but are not limited to, naturally derived compounds, such as hesperetin, hesperitin dihydrochalcone, hesperitin dihydrochalcone glucoside, 3-hydroxybenzoic acid, phloretin, naringenin, rhoifolin, glucosylated steviol glycosides, (2R,3R)-3-acetoxy-5,7,4′-trihydroxyflavanone, (2R,3R)-3-acetoxy-5,7,3′-trihydroxy-4′-methoxyflavanone, or synthetic compounds, such as any compounds set forth in U.S. Pat. Nos. 8,541,421; 8,815,956; 9,834,544; 8,592,592; 8,877,922; 9,000,054; and 9,000,051, as well as U.S. Patent Application Publication No. 2017/0119032. In embodiments where the taste-modifying composition is used in combination with a sweetness enhancer, the concentration of PMF compounds used in combination with such sweetness enhancers has a w/w ratio ranging from 1:1000 to 1000:1, or from 1:100 to 100:1, or from, 1:50 to 50:1, or from 1:25 to 25:1, or from 1:10 to 10:1, such as 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1. In some embodiments of any of the preceding embodiments, the taste-modifying composition is combined with glucosylated steviol glycosides in any of the above ratios. As used herein, the term “glucosylated steviol glycoside” refers to the product of enzymatically glucosylating natural steviol glycoside compounds. The glucosylation generally occurs through a glycosidic bond, such as an α-1,2 bond, an α-1,4 bond, an α-1.6 bond, a β-1,2 bond, a β-1,4 bond, a β-1,6 bond, and so forth. In some embodiments of any of the preceding embodiments, the TM1 compound (or any comestibly acceptable salts thereof) is combined with N-(1-((4-amino-2,2-dioxo-1H-benzo[c][1,2,6]thiadiazin-5-yl)oxy)-2-methyl-propan-2-yl)isonicotinamide, in any of the above ratios.

In a similar way, the taste-modifying composition is also used, in some embodiments, in combination with umami enhancers, cooling (TRPM8) enhancers, bitterness blockers, sour taste modulators, mouthfeel enhancers, flavor-masking compounds, or any combination thereof.

In some aspects related to the preceding aspects and embodiments, the disclosure provides uses of the taste-modifying compositions disclosed herein to enhance the flavor of a flavored composition, such as a flavored article. Such flavored compositions can use any suitable flavors, such as any of the flavors set forth above.

### Flavored Products and Concentrates

In certain aspects, the disclosure provides flavored products comprising any ingestible compositions of the preceding aspects. In some embodiments, the flavored products are beverage products, such as soda, flavored water, tea, and the like. In some other embodiments, the flavored products are food products, such as yogurt.

In embodiments where the flavored product is a beverage, the beverage may be selected from the group consisting of enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts and smoothies. In some embodiments, the beverage may be a soft drink.

In certain embodiments of any aspects and embodiments set forth herein that refer to an flavored product, the flavored product is a non-naturally-occurring product, such as a packaged food or beverage product.

Further non-limiting examples of food and beverage products or formulations include sweet coatings, frostings, or glazes for such products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionery category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consommé) to sauces (cream or cheese-based soups).

The Dehydrated and Culinary Food Category usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to beer, cider/perry, FABs, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks;

concentrates, such as liquid and powder concentrates in ready to drink measure. The drinks, either hot or cold, include, but are not limited to coffee or ice coffee, such as fresh, instant, and combined coffee; tea or ice tea, such as black, green, white, oolong, and flavored tea; and other drinks including flavor-, malt-or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream and sugar and flavoring. Examples of ice cream include, but are not limited to: impulse ice cream; take-home ice cream; frozen yoghurt and artisanal ice cream; soy, oat, bean (e.g., red bean and mung bean), and rice-based ice creams.

The Confectionery category generally refers to edible product that is sweet to the taste. Examples of confectionery include, but are not limited to candies, gelatins, chocolate confectionery, sugar confectionery, gum, and the likes and any combination products.

The Meal Replacement category generally refers to any food intended to replace the normal meals, particularly for people having health or fitness concerns. Examples of meal replacement include, but are not limited to slimming products and convalescence products.

The Ready Meal category generally refers to any food that can be served as meal without extensive preparation or processing. The ready meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles. The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to milk-or soybean-based formula; and prepared, dried and other baby food.

The Spreads category includes, but is not limited to jams and preserves, honey, chocolate spreads, nut based spreads, and yeast based spreads.

The Dairy Product category generally refers to edible product produced from mammal's milk. Examples of dairy product include, but are not limited to drinking milk products, cheese, yoghurt and sour milk drinks, and other dairy products.

Additional examples for flavored products, particularly food and beverage products or formulations, are provided as follows. Exemplary ingestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads. Exemplary ingestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof. Exemplary ingestible compositions also include breakfast cereals, sweet beverages or solid or liquid concentrate compositions for preparing beverages, ideally so as to enable the reduction in concentration of previously known saccharide sweeteners, or artificial sweeteners.

Some embodiments provide a chewable composition that may or may not be intended to be swallowed. In some embodiments, the chewable composition may be gum, chewing gum, sugarized gum, sugar-free gum, functional gum, bubble gum including compounds as disclosed and described herein, individually or in combination.

In some embodiments, the taste-modifying compositions disclosed and described herein, individually or in combination, may be provided in a flavoring concentrate formulation, e.g., suitable for subsequent processing to produce a ready-to-use (i.e., ready-to-serve) product. By “a flavoring concentrate formulation”, it is meant a formulation which should be reconstituted with one or more diluting medium to become a ready-to-use composition. The term “ready-to-use composition” is used herein interchangeably with “ingestible composition”, which denotes any substance that, either alone or together with another substance, can be taken by mouth whether intended for consumption or not. In one embodiment, the ready-to-use composition includes a composition that can be directly consumed by a human or animal. The flavoring concentrate formulation is typically used by mixing with or diluted by one or more diluting medium, e.g., any consumable or ingestible ingredient or product, to impart or modify one or more flavors to the diluting medium. Such a use process is often referred to as reconstitution. The reconstitution can be conducted in a household setting or an industrial setting. For example, a frozen fruit juice concentrate can be reconstituted with water or other aqueous medium by a consumer in a kitchen to obtain the ready-to-use fruit juice beverage. In another example, a soft drink syrup concentrate can be reconstituted with water or other aqueous medium by a manufacturer in large industrial scales to produce the ready-to-use soft drinks. Since the flavoring concentrate formulation has the flavoring agent or flavor modifying agent in a concentration higher than the ready-to-use composition, the flavoring concentrate formulation is typically not suitable for being consumed directly without reconstitution. There are many benefits of using and producing a flavoring concentrate formulation. For example, one benefit is the reduction in weight and volume for transportation as the flavoring concentrate formulation can be reconstituted at the time of usage by the addition of suitable solvent, solid or liquid.

The flavored products set forth according to any of the foregoing embodiments, also include, in certain embodiments, one or more additional flavor-modifying compounds, such as compounds that enhance sweetness, compounds that block bitterness, compounds that enhance umami, compounds that reduce sourness, compounds that enhance saltiness, compounds that enhance a cooling effect, or any combinations of the foregoing.

In certain embodiments of any aspects and embodiments set forth herein that refer to a sweetening or flavoring concentrate, the sweetening or flavoring concentrate is a non-naturally-occurring product, such as a composition specifically manufactured for the production of a flavored product, such as food or beverage product.

In one embodiment, the flavoring concentrate formulation comprises i) compounds as disclosed and described herein, individually or in combination; ii) a carrier; and iii) optionally at least one adjuvant. The term “carrier” denotes a usually inactive accessory substance, such as solvents, binders, or other inert medium, which is used in combination with the present compound and one or more optional adjuvants to form the formulation. For example, water or starch can be a carrier for a flavoring concentrate formulation. In some embodiments, the carrier is the same as the diluting medium for reconstituting the flavoring concentrate formulation; and in other embodiments, the carrier is different from the diluting medium. The term “carrier” as used herein includes, but is not limited to, ingestibly acceptable carrier.

The term “adjuvant” denotes an additive which supplements, stabilizes, maintains, or enhances the intended function or effectiveness of the active ingredient, such as the compound of the present invention. In one embodiment, the at least one adjuvant comprises one or more flavoring agents. The flavoring agent may be of any flavor known to one skilled in the art or consumers, such as the flavor of chocolate, coffee, tea, mocha, French vanilla, peanut butter, chai, or combinations thereof. In another embodiment, the at least one adjuvant comprises one or more sweeteners. The one or more sweeteners can be any of the sweeteners described in this application. In another embodiment, the at least one adjuvant comprises one or more ingredients selected from the group consisting of a emulsifier, a stabilizer, an antimicrobial preservative, an antioxidant, vitamins, minerals, fats, starches, protein concentrates and isolates, salts, and combinations thereof. Examples of emulsifiers, stabilizers, antimicrobial preservatives, antioxidants, vitamins, minerals, fats, starches, protein concentrates and isolates, and salts are described in U.S. Pat. No. 6,468,576, the content of which is hereby incorporated by reference in its entirety for all purposes.

In one embodiment, the present flavoring concentrate formulation can be in a form selected from the group consisting of liquid including solution and suspension, solid, foamy material, paste, gel, cream, and a combination thereof, such as a liquid containing certain amount of solid contents. In one embodiment, the flavoring concentrate formulation is in form of a liquid including aqueous-based and nonaqueous-based. In some embodiments, the present flavoring concentrate formulation can be carbonated or non-carbonated.

The flavoring concentrate formulation may further comprise a freezing point depressant, nucleating agent, or both as the at least one adjuvant. The freezing point depressant is an ingestibly acceptable compound or agent which can depress the freezing point of a liquid or solvent to which the compound or agent is added. That is, a liquid or solution containing the freezing point depressant has a lower freezing point than the liquid or solvent without the freezing point depressant. In addition to depress the onset freezing point, the freezing point depressant may also lower the water activity of the flavoring concentrate formulation. The examples of the freezing point depressant include, but are not limited to, carbohydrates, oils, ethyl alcohol, polyol, e.g., glycerol, and combinations thereof. The nucleating agent denotes an ingestibly acceptable compound or agent which is able to facilitate nucleation. The presence of nucleating agent in the flavoring concentrate formulation can improve the mouthfeel of the frozen Blushes of a frozen slush and to help maintain the physical properties and performance of the slush at freezing temperatures by increasing the number of desirable ice crystallization centers. Examples of nucleating agents include, but are not limited to, calcium silicate, calcium carbonate, titanium dioxide, and combinations thereof.

In one embodiment, the flavoring concentrate formulation is formulated to have a low water activity for extended shelf life. Water activity is the ratio of the vapor pressure of water in a formulation to the vapor pressure of pure water at the same temperature. In one embodiment, the flavoring concentrate formulation has a water activity of less than about 0.85. In another embodiment, the flavoring concentrate formulation has a water activity of less than about 0.80. In another embodiment, the flavoring concentrate formulation has a water activity of less than about 0.75.

In one embodiment, the flavoring concentrate formulation has the present compound in a concentration that is at least 2 times of the concentration of the compound in a ready-to-use composition. In one embodiment, the flavoring concentrate formulation has the present compound in a concentration that is at least 5 times of the concentration of the compound in a ready-to-use composition. In one embodiment, the flavoring concentrate formulation has the present compound in a concentration that is at least 10 times of the concentration of the compound in a ready-to-use composition. In one embodiment, the flavoring concentrate formulation has the present compound in a concentration that is at least 15 times of the concentration of the compound in a ready-to-use composition. In one embodiment, the flavoring concentrate formulation has the present compound in a concentration that is at least 20 times of the concentration of the compound in a ready-to-use composition. In one embodiment, the flavoring concentrate formulation has the present compound in a concentration that is at least 30 times of the concentration of the compound in a ready-to-use composition. In one embodiment, the flavoring concentrate formulation has the present compound in a concentration that is at least 40 times of the concentration of the compound in a ready-to-use composition. In one embodiment, the flavoring concentrate formulation has the present compound in a concentration that is at least 50 times of the concentration of the compound in a ready-to-use composition. In one embodiment, the flavoring concentrate formulation has the present compound in a concentration that is at least 60 times of the concentration of the compound in a ready-to-use composition. In one embodiment, the flavoring concentrate formulation has the present compound in a concentration that is up to 100 times of the concentration of the compound in a ready-to-use composition.

### Treatment of Compositions of Diseased Citrus

In one or more aspects, the disclosure provides methods for reducing the bitterness of citrus compositions derived from citrus fruit having citrus greening disease, the method comprising: (a) deriving a citrus composition from citrus fruit, wherein at least a portion of the citrus fruit has citrus greening disease; and (b) introducing to the citrus composition a bitterness-reducing composition, which comprises a taste-modifying composition of any of the foregoing aspects or embodiments, such as an amount having a bitterness-reducing effective amount of PMF compounds.

In some embodiments, the citrus composition comprises an abnormally elevated concentration of one or more bitter compounds, such as abnormally elevated levels of limonin or nomilin. In some embodiments, the bitter compounds are limonoids, such as limonin, nomilin, nomilinic acid, or any combination thereof.

### Blocking Bitterness in Pharmaceutical APIs

Many drug compounds impart a bitter taste, which therefore limits the ways in which they can be formulated and administered. Therefore, in certain aspects, the disclosure provides a pharmaceutical composition comprising a bitter-tasting pharmaceutical active ingredient and a taste-modifying composition of any of the foregoing aspects and embodiments. Such pharmaceutical compositions can be in any suitable form for oral administration, such as tablets, lozenges, capsules, powders, liquid solutions, liquid suspensions, and the like. Such pharmaceutical compositions can include any suitable pharmaceutical excipients, binders, and the like, such as those set forth in Remington's Pharmaceutical Sciences. In some embodiments, the bitter-tasting pharmaceutical active ingredient is an ion channel inhibitor, such as a proton channel inhibitor. Other examples of bitter-tasting APIs whose bitterness is reduced by the taste-modifying compositions disclosed herein include, but are not limited to, atropine, brinzolamide, chloramphenicol, chloroquine, clindamycin, dexamethasone, digoxin, diltiazem, diphenhydramine, docusate, dorzolamide, doxepin, doxylamine, enalapril, erythromycin, esomeprazole, famotidine, gabapentin, ginkgolide A, guaifenesin, L-histidine, lomefloxacin, methylprednisolone, ofloxacin, oleuropein, oxyphenonium, pirenzepine, prednisone, ranitidine, trapidil, trimethoprim, and cetirizine.

### Non-Animal Protein Materials and Products Made Therefrom

Products intended to replace or substitute meat or dairy products often rely on various non-animal-based materials, such as starches, fibers, and proteins derived from plants, algae, fungi, or combinations thereof, to simulate the texture and flavor of meat or dairy. Non-limiting examples of non-animal-based proteins are plant proteins, such as soy proteins, pea proteins, bean proteins, grain proteins, potato protein, and the like. Due to compositional differences between such non-animal-based materials and animal-derived materials, these products can present a bitter taste that consumers may find off-putting.

Thus, in certain aspects, the disclosure provides a flavored product comprising a plant-based material (such as a plant-based starch, a plant-based fiber, a plant-based protein, or a combination thereof) and a taste-modifying composition disclosed herein. In some further embodiments, the flavored product can include any features of combination of features set forth above for ingestible compositions that contain the taste-modifying composition. In some embodiments, the flavored product is a beverage, such as soy milk, almond milk, rice milk, oat milk, a protein drink, a meal-replacement drink, or other like product. In some other embodiments, the flavored product is a meat-replacement product, such as a plant-based chicken product (such as a plant-based chicken nugget), a plant-based beef product (such as a plant-based burger), and the like. In some other embodiments, the flavored product is a protein powder, a meal-replacement powder, a plant-based creamer for coffee or tea, and the like. In certain further embodiments, any such products contain additional ingredients, and have additional features, as are typically used in the preparation and/or manufacture of such products. For example, such the taste-modifying composition may be combined with other flavors and taste modifiers, and may even be encapsulated in certain materials, according to known technologies in the relevant art. Suitable concentrations of the taste-modifying composition are set forth above.

In some embodiments, the flavored products comprise one or more plant-based proteins, which impart a bitter taste that is at least partially reduced by the use of the taste-modifying composition in the product. Such plant-based proteins include, but are not limited to, pea protein, soy protein, canola (rapeseed) protein, chickpea protein, mycoproteins, algal proteins, fava protein, sunflower protein, wheat protein,

In some alternative embodiments analogous to the above embodiments, algal or fungal proteins or starches are used instead, oat protein, potato protein, and the like. In some embodiments, these flavored products also include fiber to provide texture to the product. Fibers suitable for use include, but are not limited to, psyllium fiber, pea fiber, potato fiber, curdlan, soluble corn fiber (dextran and/or maltodextrin), citrus fiber, and combinations thereof. In such products, the taste-modifying composition can be introduced in any suitable way. In some embodiments, the taste-modifying composition is incorporated into a flavoring emulsion, such as a water-in-oil emulsion, along with other flavor-imparting ingredients.

### Non-Meat Protein Materials and Products Made Therefrom

Certain non-meat animal proteins, such as dairy proteins and proteins from bone broth, are commonly used in food products, and are also sold as the primary ingredient in certain protein powders. Such proteins can impart bitter flavors that consumers may not desire. This is especially true for protein isolates, such as protein isolates of whey protein, collagen protein, casein proteins, and the like. Thus, the present disclosure provides ingestible compositions that include non-meat animal proteins and the taste-modifying composition. In some embodiments, the non-meat animal protein is a bone protein, such as a collagen protein derived from the bones of an animal, such as a cow, pig, donkey, horse, chicken, duck, goat, goose, rabbit, lamb, sheep, buffalo, ostrich, camel, and the like. In some embodiments, the non-meat animal protein is a milk protein, such as a whey protein, a casein protein, or any combination thereof. The milk can be the milk of any suitable animal, such as a cow, donkey, horse, sheep, buffalo, camel, and the like.

The taste-modifying composition can also be included in certain food or beverage products that include animal milk or materials derived from animal milk. Such products include cheeses, cheese spreads, yogurt, kefir, milk, processed dairy products, cottage cheese, sour cream, butter, and the like.

### EXAMPLES

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### Example 1—Synthetic Processes

### Step 1—Extraction

Citrus Valencia peel oil distillated residues (490.4 g) was mixed into 50% (w/w) aqueous ethanol (2500 g) by stirring at 350 rpm for 3 hours. The ethanol aqueous extract solution upper layer was separated from the bottom sediment part and went through a membrane filter (0.45 μm) to filter out the insoluble components. An extract solution of 2650 grams was obtained.

A portion of extract solution (100.0 g) was concentrated using a rotary evaporator and further freeze-dried in a freeze-dryer. The dried extract (3.2 g) was stored at 4° C. for chemical component analysis. This sample is referred to as Sample 1A

### Step 2—Adsorption Chromatography

The adsorption chromatography purification of the extracted flavonoids was conducted using a flash chromatography system (Agela Technologies, FLEXA) equipped with a flash column packed with macroporous resin or MCI gel. The resins were first activated with 95% ethanol and then conditioned with deionized water.

Then, 300 g of the the extract solution from step 1 was loaded onto the top of the flash column (80 g, 150 mL, I.D. 3.12 cm×H. 19.5 cm) packed with MCI gel (Mitsubishi Chemical Corp., CHP20) at a flow rate 5 mL/min. After adsorption, the desorption was conducted using 50% of aqueous ethanol (w/w). The constant flow rate of the mobile phase was 10 mL/min. The elution volume was kept at 8 column volumes. The collected fraction (1st through 8th bed volumes) was concentrated using a rotary evaporator and then dried using freeze-dryer. The dried product was stored at 4° C. for chemical components analysis. This sample is referred to as Sample 2A.

Then, 300 g of the extract solution from step 1 was loaded onto the top of the flash column (120 g, 215 mL, I.D. 3.66 cm×H. 20.4 cm) packed with AB-8 resin at flow rate 5 mL/min. After adsorption, the desorption was conducted was conducted using 50% of aqueous ethanol (w/w). The constant flow rate of the mobile phase was 5 mL/min. The elution volume was kept at 10 column volumes. The collected fraction (1st through 10th bed volumes) was concentrated using a rotary evaporator and then dried using freeze-dryer. The dried product was stored at 4° C. for chemical components analysis. This sample is referred to as Sample 2B.

Then, 300 g of the extract solution from step 1 was loaded onto the top of the flash column (120 g, 215 mL, I.D. 3.66 cm×H. 20.4 cm) packed with D101 resin at flow rate 5 mL/min. After adsorption, the desorption was conducted using 50% of aqueous ethanol (w/w). The constant flow rate of the mobile phase was 10 mL/min. The elution volume was kept at 10 column volumes. The collected fraction (1st through 10th bed volumes) was concentrated using a rotary evaporator and then dried using freeze-dryer. The dried product was stored at 4° C. for chemical components analysis. This sample is referred to as Sample 2C-1

The D101 resin after first cycle separation was regenerated with diluted sodium hydroxide solution (0.5% w/v) and then with 95% ethanol. The ethanol was thoroughly washed with deionized water. Then, 300 g of the extract solution from step 1 was loaded onto the top of the flash column packed with D101 resin (2nd cycle separation) at flow rate 5 mL/min. After adsorption, the desorption was conducted using 50% of aqueous ethanol (w/w). The constant flow rate of the mobile phase was 10 mL/min. The elution volume was kept at 10 column volumes. The collected fraction (1st through 10th bed volumes) was concentrated using a rotary evaporator and then dried using freeze-dryer. The dried product was stored at 4° C. for chemical components analysis. This sample is referred to as Sample 2C-2.

Then, 300 g of the extract solution from step 1 was loaded onto the top of the flash column (120 g, 215 mL, I.D. 3.66 cm×H. 20.4 cm) packed with HP20 resin at flow rate 5 mL/min. After adsorption, the desorption was conducted using 50% of aqueous ethanol (w/w). The constant flow rate of the mobile phase was 10 mL/min. The elution volume was kept at 10 column volumes. The collected fraction (1st through 10th bed volumes) was concentrated using a rotary evaporator and then dried using freeze-dryer. The dried product was stored at 4° C. for chemical components analysis. This sample is referred to as Sample 2D-1.

The HP20 resin after the first cycle separation was regenerated with 95% of aqueous ethanol (w/w) and pure acetone. Acetone was thoroughly washed with 95% of aqueous ethanol (w/w). The column conditioned with deionized water. Then, 300 g of the extract from step 1 was loaded onto the top of the flash column packed with HP20 resin (2nd cycle separation) at flow rate 5 mL/min. After adsorption, the desorption was conducted using 50% of aqueous ethanol (w/w). The constant flow rate of the mobile phase was 10 mL/min. The elution volume was kept at 10 column volumes. The collected fraction (2nd through 10th bed volumes) was concentrated using a rotary evaporator and then dried using freeze-dryer. The dried product was stored at 4° C. for chemical components analysis. This sample is referred to as Sample 2D-2.

The HP20 resin after the second cycle separation was regenerated with 95% of aqueous ethanol (w/w) and pure acetone. Acetone was thoroughly washed with 95% of aqueous ethanol (w/w). The column conditioned with deionized water. Then, 300 g of the extract from step 1 was loaded onto the top of the flash column packed with HP20 resin (3rd cycle separation) at flow rate 5 mL/min. After adsorption, the desorption was conducted using 50% of aqueous ethanol (w/w). The constant flow rate of the mobile phase was 10 mL/min. The elution volume was kept at 10 column volumes. The collected fraction (2nd through 10th bed volumes) was concentrated using a rotary evaporator and then dried using freeze-dryer. The dried product was stored at 4° C. for chemical components analysis. This sample is referred to as Sample 2D-3.

The HP20 resin after the third cycle separation was regenerated with 95% of aqueous ethanol (w/w) and pure acetone. Acetone was thoroughly washed with 95% of aqueous ethanol (w/w). The column conditioned with deionized water. Then, 300 g of the extract derived from another batch of orange peel oil distillated residue was loaded onto the top of the flash column packed with HP20 resin (4th cycle separation) at flow rate 5 mL/min. After adsorption, the desorption was conducted firstly using 50% of aqueous ethanol (w/w) and secondary using 65% of aqueous ethanol (w/w). The constant flow rate of the mobile phase was 10 mL/min. The elution volume was kept at 10 column volumes. The collected fractions (2nd through 10th bed volumes of 50% and 1st through 10th bed volumes of 65%) were concentrated using a rotary evaporator and then dried using freeze-dryer. The dried product (2nd through 10th bed volumes of 50%), referred to as Sample 2D-4.

Then, 300 g of the the extract solution from step 1 was loaded onto the top of the flash column (120 g, 215 mL, I.D. 3.66 cm×H. 20.4 cm) packed with XAD1600 resin at flow rate 5 mL/min. After adsorption, the desorption was conducted using 50% of aqueous ethanol (w/w). The constant flow rate of the mobile phase was 10 mL/min. The elution volume was kept at 10 column volumes. The collected fraction (1st through 10th bed volumes) was concentrated using a rotary evaporator and then dried using freeze-dryer. The dried product was stored at 4° C. for chemical components analysis. This sample is referred to as Sample 2E.

Then, 300 g of the extract solution from step 1 was loaded onto the top of the flash column (120 g, 215 mL, I.D. 3.66 cm×H. 20.4 cm) packed with XAD4 resin at flow rate 5 mL/min. After adsorption, the desorption was conducted using different concentrations of aqueous ethanol successively (50%, 55% and 65%). The constant flow rate of the mobile phase was 10 mL/min. The elution volume was kept at 10 column volumes for 50% aqueous ethanol eluent and 5 column volumes for 55% and 65% aqueous ethanol eluent. The collected fractions (1st through 10th bed volumes of 50% and 1st through 5th bed volumes of 55%) were concentrated using a rotary evaporator and then dried using freeze-dryer. The dried product was stored at 4° C. for chemical components analysis, which is referred to as Sample 2F.

### Step 3—Base Wash

The dried product from step 2 (Sample 2D-2) was treated with a dilute sodium hydroxide (5% w/v) by stirring at 350 rpm, 80° C. for 2hours. An insoluble component was collected, dissolved in ethyl acetate, and washed using deionized water twice, then dried to form a product (Sample 3A).

Citrus Pera oil distillated residue (518.11 g) was mixed into 50% (w/w) aqueous ethanol (2590 g) by stirring at 350 rpm, at room temperature for 3 hours. The ethanol aqueous extract solution upper layer was separated from the bottom sediment part and went through a membrane filter (0.25 μm) to filter out the insoluble components. Then, 2590 g of extract solution was obtained. A small part of extract solution (100.0 g) was first concentrated using a rotary evaporator and further freeze-dried in a freeze-dryer. The dried extract (Sample 4A, 3.225 g) was stored at 4° C. for chemical component analysis. Then, 300 g of the extract solution was loaded onto the top of the flash column (220 g, 377 mL, I.D. 60.6 mm×H. 13.1 mm) packed with HP20 resin at flow rate 10 mL/min. After adsorption, the desorption was conducted using 50% of aqueous ethanol (w/w). The constant flow rate of the mobile phase was 20 mL/min. The elution volume was kept at 10 column volumes. The collected fraction (2nd through 10th bed volumes) was concentrated using a rotary evaporator and then dried using freeze-dryer. Then, 100 mg of dried product (Sample 5D-1) was stored at 4° C. for chemical components analysis. The rest of the dried product of Sample 5D-1 was treated with a dilute sodium hydroxide (5% w/v) by stirring at 350 rpm, 80° C. for 2 hours. An insoluble component was collected, dissolved in ethyl acetate, and washed using deionized water twice, then dried to form a product (Sample 6A).

### Analytical Method

The PMFs were mainly composed of the six PMFs set forth in Table 1. These individual six PMFs were isolated from orange peel oil distillated residues were used as external standards, their structures were identified as sinensetin (SIN, CAS No. 2306-27-6), hexa-O-methylquercetagetin (HMQ, CAS No. 1251-84-9), nobiletin (NOB, CAS No. 478-01-3), TOM-scutellarein (TMS, CAS No. 1168-42-9), heptamethoxyflavone (HMF, CAS No. 1178-24-1), and tangeretin (TAN, CAS No. 481-53-8), and their purity (above 95%) were confirmed by NMR and LCMS analysis. The main components of hydroxy-substituted flavonoids were their demethyl derivatives. Six of them were isolated from the mixture and determined to be 5-demethyl sinensetin (5OHSIN, CAS No. 21763-80-4), 5-demethyl HMQ (5OHHMQ, CAS No. 479-90-3), 5-demethyl nobiletin (5OHNOB, CAS No. 2174-59-6), 5-demethyl TMS (5OHTMS, CAS No. 19103-54-9), 5-demethyl heptamethoxyflavone (5OHHMF, CAS No. 1176-88-1), and 5-demethyl tangeretin (5OHTAN, CAS No. 2798-20-1). Other minor products were determined to be their hydroxy-or methoxy-isomers. A reverse phase method with an RP18 column (Luna C18(2), 150*2.0 mm*3 μm) and UV detector was used to quantitively analyze the products. The calibration curves were obtained by running multi-standard solutions on Agilent UPLC system equipped with PDA detector. The flavonoids contents in all samples on dry basis were determined, and are shown in Table 2.

TABLE 2

| | | | | | | | | PMF % in |
|---|---|---|---|---|---|---|---|---|
| | | | | | | | | total |
| Sample | SIN | HMQ | NOB | TMS | HMF | TAN | OHPMF* | flavonoids |
| Orange | 0.75% | 0.80% | 4.05% | 2.07% | 3.50% | 2.80% | 1.62% | 89.5% |
| peel oil | | | | | | | | |
| distilled | | | | | | | | |
| residue | | | | | | | | |
| 1A | 3.2% | 3.7% | 19.7% | 9.4% | 16.9% | 13.1% | 6.3% | 91.3% |
| 2A | 4.6% | 5.3% | 28.3% | 12.8% | 23.0% | 12.4% | 4.3% | 95.3% |
| 2B | 4.7% | 5.2% | 27.4% | 11.1% | 21.2% | 11.1% | 4.6% | 94.6% |
| 2C-1 | 4.2% | 4.9% | 26.0% | 11.7% | 21.6% | 13.3% | 5.5% | 93.7% |
| 2C-2 | 4.2% | 4.9% | 26.1% | 11.7% | 21.6% | 13.1% | 5.3% | 93.9% |
| 2D-1 | 4.8% | 5.4% | 28.6% | 11.9% | 22.4% | 10.4% | 4.1% | 95.4% |
| 2D-2 | 5.1% | 5.7% | 30.2% | 12.3% | 23.5% | 10.5% | 4.1% | 95.4% |
| 2D-3 | 4.5% | 6.0% | 30.0% | 13.4% | 24.5% | 9.8% | 5.1% | 94.5% |
| 2D-4 | 5.9% | 29.5% | 15.8% | 16.9% | 13.1% | 5.9% | 6.3% | 93.2% |
| 2E | 4.5% | 5.2% | 27.4% | 11.7% | 22.3% | 11.4% | 4.3% | 95.0% |
| 2F | 5.0% | 5.4% | 28.5% | 11.2% | 21.5% | 10.8% | 4.2% | 95.2% |
| 3A | 5.3% | 6.3% | 30.6% | 13.5% | 25.3% | 11.1% | 4.8% | 95.0% |
| Citrus | 0.4% | 0.5% | 3.3% | 1.5% | 3.6% | 2.2% | 2.0% | 85.2% |
| Pera oil | | | | | | | | |
| distillated | | | | | | | | |
| residue | | | | | | | | |
| 4A | 2.7% | 2.6% | 19.6% | 8.7% | 19.6% | 11.7% | 6.1% | 91.4% |
| 5D-1 | 4.5% | 6.0% | 29.9% | 13.4% | 24.6% | 9.8% | 6.5% | 93.1% |
| 6A | 4.6% | 6.2% | 30.5% | 13.8% | 25.4% | 10.2% | 5.7% | 94.1% |

### Example 2—Sensory Testing

Certain PMF compositions were tasted by a human sensory panel to assess their off note potency. The panel consisted of 7 trained panelists evaluating the samples at 5 ppm concentration on a scale of 0 to 10 (with 0 indicating no off notes, and 10 indicating strong off notes). The results are set forth in Table 3. The off-note intensity was decreased after each step of the process.

TABLE 3

| Sample | Off Note Potency |
|---|---|
| 1A | 4.9 |
| 2C-2 | 3.6 |
| 3A | 1.7 |

1. A process for concentrating polymethoxylated flavonoids, the process comprising:
(a) providing an organic oil, which comprises polymethoxylated flavonoids and hydroxyl-substituted flavonoids, and wherein the concentration of polymethoxylated flavonoids in the organic oil is no more than 30% by weight, based on the total weight of the organic oil;
(b) introducing an extraction medium to the organic oil to form a mixture having an aqueous phase and an organic phase, wherein the extraction medium comprises water and a C₁₋₆ alcohol;
(c) separating at least a portion of the aqueous phase from the organic phase;
(d) generating two or more fractions of the separated aqueous phase using adsorption chromatography, wherein at least one of the two or more fractions comprises polymethoxylated flavonoids or both polymethoxylated flavonoids and hydroxyl-substituted flavonoids at a concentration of at least 85% by weight, on a dry weight basis; and
(e) washing the at least one of the two or more fractions with an aqueous alkaline solution.

2. The process of claim 1, wherein the polymethoxylated flavonoids are flavones.

3. The process of claim 2, wherein the polymethoxylated flavonoids are selected from the group consisting of sinensetin, hexamethoxyquercetagetin, nobiletin, tetramethoxy-scutellarein, heptamethoxyflavone, tangeretin, and any combinations thereof.

4. (canceled)

5. A taste-modifying composition comprising one or more polymethoxylated flavonoids and one or more hydroxyl-substituted flavonoids, wherein the composition comprises polymethoxylated flavonoids or both polymethoxylated flavonoids and hydroxyl-substituted flavonoids at a concentration of at least 85% by weight, on a dry weight basis.

6. The taste-modifying composition of claim 5, wherein the polymethoxylated flavonoids are flavones.

7. The taste-modifying composition of claim 6, wherein the polymethoxylated flavonoids are selected from the group consisting of sinensetin, hexamethoxyquercetagetin, nobiletin, tetramethoxy-scutellarein, heptamethoxyflavone, tangeretin, and any combinations thereof.

8. The taste-modifying composition of claim 5, wherein the weight ratio of polymethoxylated flavonoids to hydroxy-substituted flavonoids in the composition is at least 9:1.

9. A method of reducing a bitter taste or an astringency of an ingestible composition, the method comprising introducing to the ingestible composition a taste modifying composition of claim 5.

10. An ingestible composition, which comprises a taste-modifying composition of claim 5.

11. A flavored product, which comprises an ingestible composition of claim 10.

12. The flavored product of claim 11, wherein the flavored product is a food or beverage product.
